(19) United States
(12) Patent Application Publication
(10) Pub. No.: US 2025/0093784 A1
(43) Pub. Date: Mar. 20, 2025

(54) APPARATUS AND METHOD FOR ASSESSING PHOTORESIST RINSE SOLUTION

(71) Applicant: SAMSUNG ELECTRONICS CO., LTD., SUWON-SI (KR)

(72) Inventors: DAIKI MINAMI, Suwon-si (KR); JINJOO KIM, Suwon-si (KR); HYUNJI SONG, Suwon-si (KR); JI YOUNG PARK, Suwon-si (KR); SEUNGYEOL BAEK, Suwon-si (KR)

(21) Appl. No.: 18/652,444

(22) Filed: May 1, 2024

(30) Foreign Application Priority Data
Sep. 15, 2023 (KR) 10-2023-0123041

(51) Int. Cl. G03F 7/00; G03F 7/40; G06F 30/20

(52) U.S. Cl. CPC G03F 7/705; G03F 7/40; G03F 7/70508; G06F 30/20

(57) ABSTRACT

An apparatus includes a processor, a memory electrically connected to the processor and that stores instructions, and a storage unit that stores a photoresist molecular model, a surfactant molecular model, an additive molecular model and a solvent molecular model. The processor is configured by the instructions to perform a molecular dynamics simulation of a cell in which are placed photoresist molecules based on the photoresist molecular model, surfactant molecules based on the surfactant molecular model, additive molecules based on the additive molecular model, and solvent molecules based on the solvent molecular model, and compute a number of first surfactant molecules located in the cell in a vicinity of an interface between a photoresist pattern that includes the photoresist molecules and a solvent that includes the solvent molecules, and a number of second surfactant molecules located in the cell in a vicinity of a surface of the solvent.

### CROSS-REFERENCE TO RELATED APPLICATIONS

This application claims priority under 35 U.S.C. § 119 from Korean Patent Application No. 10-2023-0123041, filed on Sep. 15, 2023 in the Korean Intellectual Property Office, the contents of which are herein incorporated by reference in their entirety.

### TECHNICAL FIELD

Embodiments of the present disclosure described herein are directed to a method and an apparatus for assessing a photoresist rinse solution based on a molecular dynamics simulation, and a method and an apparatus for simulating a lithography process based on a simulation of a photoresist rinse solution.

### DISCUSSION OF THE RELATED ART

In a semiconductor device manufacturing process, microfabrication can be performed by a lithography process that uses a photoresist composition. Due to a high level of integration of integrated circuits, ultra-fine patterns are formed in very small areas. With a high level of integration, a wavelength of light used in the lithography is also becoming shorter. Recently, a lithography technology that uses extreme ultraviolet light (EUV) in addition to deep ultraviolet light (DUV) has been used.

In a lithography process, after a photoresist (PR) composition that is a photosensitizer is applied to the wafer, exposure and development processes are performed. After the development process, a rinse process is performed with a rinse solution to clean resist film. When the ingredients of the rinse solution are inappropriate, the photoresist pattern formed on the wafer can collapse.

### SUMMARY

Embodiments of the present disclosure provide a method and an apparatus that assesses a rinse solution that prevents collapse of a photoresist and is suitable for a rinse process, and a method and an apparatus for simulating a lithography process.

An apparatus for assessing a photoresist rinse solution includes a processor, a memory electrically connected to the processor and that stores instructions performed by the processor, and a storage unit that stores a photoresist molecular model, a surfactant molecular model, an additive molecular model and a solvent molecular model. The processor is configured by the instructions to perform a molecular dynamics simulation of a cell in which are placed photoresist molecules based on the photoresist molecular model, surfactant molecules based on the surfactant molecular model, additive molecules based on the additive molecular model, and a solvent molecules based on the solvent molecular model, and compute a number of first surfactant molecules located in the cell in a vicinity of an interface between a photoresist pattern that includes the photoresist molecules and a solvent that includes the solvent molecules, and the number of second surfactant molecules located in the cell in a vicinity of a surface of the solvent.

A method for assessing a photoresist rinse method that is performed by a computing device includes performing a molecular dynamics simulation of a cell, in which are place photoresist molecules, surfactant molecules, additive molecules, and solvent molecules, and assessing a number of first surfactant molecules located on an interface between a photoresist pattern based on the photoresist molecules and a solvent based on the solvent molecules, and a number of second surfactant molecules located on a surface of the solvent.

A lithography process simulation method that is performed by a computing device includes receiving a mask layout and a lithography model, simulating a photoresist pattern on a wafer based on the mask layout and the lithography model, simulating a deionized water washing process on the photoresist pattern, simulating a rinse process on the photoresist pattern, and acquiring surface structure data on one area of the wafer based on the photoresist pattern on which the rinse process is simulated and the lithography model. Simulating the rinse process includes forming a cell that includes photoresist molecules based on the photoresist pattern, placing surfactant moles, additive molecules, and solvent molecules in the cell, and performing a molecule dynamics simulation of the cell, and simulating a change in the photoresist pattern based on a number of first surfactant molecules located on an interface between a photoresist pattern based on the photoresist molecules and a solvent based on the solvent molecules, and a number of second surfactant molecules located on a surface of the solvent.

### BRIEF DESCRIPTION OF THE DRAWINGS

FIG. 1 is a block diagram of an apparatus that assesses a rinse solution according to an embodiment of the present disclosure.

FIG. 2 is a block diagram of a configuration of an apparatus that assesses a rinse solution according to an embodiment of FIG. 1.

FIG. 3 is a block diagram of a simulating module of an apparatus that assesses a rinse solution according to an embodiment of FIG. 1.

FIG. 4 is a flowchart of a method of assessing a rinse solution according to an embodiment of the present disclosure.

FIG. 5 is a flowchart of a method of simulation of assessing a rinse solution according to an embodiment of FIG. 4.

FIG. 6 illustrates photoresist molecules on which a molecular dynamics simulation is performed based on a molecular model according to an embodiment of FIG. 5.

FIGS. 7A and 7B illustrate a formation of a photoresist pattern based on a molecular dynamics (MD) simulation according to an embodiment of FIG. 5.

FIG. 8 illustrates surfactant molecules on which a molecular dynamics (MD) simulation is performed based on a molecular model according to an embodiment of FIG. 5.

FIGS. 9A and 9B a molecular dynamics (MD) simulation based on an NVT ensemble of a cell according to an embodiment of FIG. 5.

FIG. 10 is a flowchart of an assessment based on a simulation result of a rinse solution in a method of assessing the rinse solution according to an embodiment of FIG. 4.

FIG. 11 illustrates first surfactant molecules and second surfactant molecules according to an embodiment of FIG. 10.

FIG. 12 illustrates first surfactant molecules and second surfactant molecules according to an embodiment of FIG. 10.

FIG. 13 illustrates an assessment reference based on a simulation result of a rinse solution according to an embodiment of FIG. 10.

FIG. 14 is a block diagram of a configuration of an apparatus that simulates a lithography process according to an embodiment of the present disclosure.

FIG. 15 is a flowchart of a method for simulating a lithography process according to an embodiment of the present disclosure.

### DETAILED DESCRIPTION

Hereinafter, embodiments of the present disclosure will be described in sufficient detail so that an ordinary person in the art to which the present disclosure pertains can easily carry out the present disclosure.

Embodiments of the present disclosure are directed to an assessment of a performance of a rinse solution used in a lithography process. The lithography process forms a fine pattern on a substrate by using a photoresist (PR), and forms a photoresist layer on the substrate by using thermal, mechanical, and chemical characteristics of the photoresist, and then exposes the photoresist layer to light of a specific wavelength and develops the exposed photoresist layer to form a photoresist pattern, and performing dry or wet etching by using the photoresist pattern as a mask. After the development, a washing process and a rinse process that uses a rinse solution is performed. Embodiments of the present disclosure provide an apparatus and a method that assesses a photoresist rinse solution to provide a rinse solution that minimizes collapse of the formed photoresist pattern.

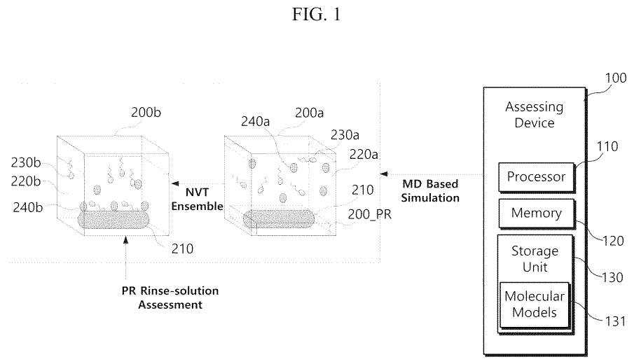

FIG. 1 is a block diagram of an apparatus that assesses a rinse solution used in a lithography process according to an embodiment of the present disclosure.

An apparatus 100 according to an embodiment of the present disclosure, hereinafter referred to as ‘a rinse solution assessing apparatus’, that assesses a rinse solution simulates behavior of molecules based on molecular dynamics (MD). The molecules constitute the rinse solution used in the rinse process of lithography. The rinse solution assessing apparatus 100 forms a photoresist pattern by performing a molecular dynamics (MD) simulation on photoresist molecules that are placed in an interior of a cell. The rinse solution assessing apparatus 100 places surfactant molecules, additive molecules, and solvent molecules in an interior of the cell separately from the photoresist pattern, and performs a molecular dynamics (MD) simulation based on an isothermal-isobaric ensemble (NPT) ensemble, and assesses the number of the surfactant molecules at a plurality of locations of the cell. The rinse solution assessing apparatus 100 assesses the rinse solution based on the number of the surfactant molecules at the plurality of locations. Performing a molecular dynamics simulation with the NPT ensemble means calculating behaviors of the molecules in an isolated system, in which the number of particles in the system, a pressure of the system, and a temperature of the system are fixed.

Referring to FIG. 1, in an embodiment, the apparatus 100 that assesses a rinse solution includes a processor 110, a memory 120, and a storage unit 130.

The memory 120 of the rinse solution assessing apparatus 100 stores instructions that control operations of the processor 110. The instructions are loaded in the memory 120 based on a code.

The storage unit 130 stores a molecular model 131 of different kinds of molecules. For example, the storage unit 130 stores a molecular model of a plurality of different kinds of photoresist materials, a molecular model of different kinds of surfactant materials, a molecular model of different kinds of additive materials, and a molecular model of different kinds of solvent materials. Accordingly, a rinse solution composition that is suitable for a specific photoresist material can be assessed while changing different kinds of photoresist materials, surfactant materials, additive materials, and/or solvent materials.

The molecular model 131 includes information related to a structure of the molecule that includes bonding relationships of the atoms. In an embodiment, the molecular model includes information related to charges of parts of the molecule.

The processor 110 loads the molecular model 131 from the storage unit 130. The processor 110 performs a molecular dynamics (MD) simulation based on the molecular model 131. For example, the processor 110 performs a molecular level simulation of individual molecules in the cell. The rinse solution assessing apparatus 100 calculates intermolecular interactions based on the molecular structure and partial charges of the molecular model 131 and simulates behaviors of the individual molecules at a molecular scale based on the intermolecular interactions.

The processor 110 of the rinse solution assessing apparatus 100 according to an embodiment of the present disclosure forms a photoresist pattern 210 by performing a molecular dynamics (MD) simulation of the photoresist molecules.

According to an embodiment, the photoresist molecules are placed in an interior of a first cell 200_PR, and a molecular dynamics (MD) simulation is performed with a molecular NPT ensemble in the first cell 200_PR to form the photoresist pattern 210. The rinse solution assessing apparatus 100 places the first cell 200_PR, in which the photoresist pattern 210 is formed, in an interior of a second cell 200a, and places solvent molecules 220a, surfactant molecules 230a, and additive molecules 240a at locations in the second cell 200a that are separated from the photoresist pattern 210. The number of the solvent molecules 220a is greater than the number of the surfactant molecules 230a and the additive molecules 240a. The rinse solution assessing apparatus 100 performs a molecular dynamics (MD) simulation of the second cell 200a, in which the solvent molecules 220a, the surfactant molecules 230a, and the additive molecules 240a are placed. According to an embodiment, the solvent molecules 220a are water molecules.

According to an embodiment, the rinse solution assessing apparatus 100 places the photoresist molecules in a specific area in the second cell 200a and forms the photoresist pattern 210 in a partial area of the second cell 200a by performing a molecular dynamics (MD) simulation. Thereafter, the rinse solution assessing apparatus 100 places the solvent molecules 220a, and surfactant molecules 230a, and the additive molecules 240a at locations in the second cell 200a that are separated formed the photoresist pattern 210. The rinse solution assessing apparatus 100 performs a molecular dynamics (MD) simulation on the cell 200a, in which the solvent molecules 220a, the surfactant molecules 230a, and the additive molecules 240a are placed.

The rinse solution assessing apparatus 100 performs a molecular dynamics (MD) simulation with a canonical (NVT) ensemble on the cell 200a, in which the photoresist pattern 210, the solvent molecules 220a, the surfactant molecules 230a, and the additive molecules 240a are placed. Performing a molecular dynamics (MD) simulation with an NVT ensemble means calculating behaviors of molecules in an isolated system, in which the number of particles in the system, a volume of the system, and a temperature of the system are fixed.

Each of the first cell 200_PR and the second cells 200a and 200b before and after the molecular dynamics (MD) simulation is performed has a constant simulation space volume that has a width, a length, and a height. The first cell 200_PR, in which the photoresist molecules are placed, and the second cell 200a, in which the solvent molecules 220a, the surfactant molecules 230a, and the additive molecules 240a are placed, have different sizes.

The processor 110 of the rinse solution assessing apparatus 100 according to an embodiment of the present disclosure computes the number of the surfactant molecules 230b at a plurality of locations of the second cell 200b, at which a molecular dynamics (MD) simulation is performed.

The rinse solution assessing apparatus 100 computes a number of first surfactant molecules in the second cell 200b located in a vicinity of an interface between the photoresist pattern 210 and the solvent 220b, and a number of second surfactant molecules located on a surface of the solvent 220b.

In an embodiment, the vicinity of the interface between the photoresist pattern 210 and the solvent 220b, in which the number of first surfactant molecules is calculated, is preset to be within a predetermined distance from the interface of the photoresist pattern 210 and the solvent 220b toward an interior of the solvent 220b.

In an embodiment, a vicinity of the surface of the solvent 220b, in which the number of second surfactant molecules is computed, is preset as an volume within a predetermined distance in a direction from the surface of the solvent 220b toward an interior of the solvent 220b.

In an embodiment, the rinse solution assessing apparatus 100 computes a surfactant content ratio of the number of second surfactant molecules to the number of first surfactant molecules.

The photoresist pattern may collapse due to a capillary effect caused by a surface tension of the rinse solution of the photoresist pattern. This collapse phenomenon of the photoresist pattern also is related to the Young's modulus of the photoresist pattern. For example, a stress that is applied to the photoresist pattern (photoresist strip) can be alleviated by lowering the surface tension using the surfactant molecules. However, if an excessive amount of surfactants is used, even though the surface tension is lowered, the collapse of the photoresist pattern may be accelerated

Accordingly, the rinse solution assessing apparatus 100 computes the number of the first surfactant molecules located in the vicinity of the interface between the photoresist pattern 210 and the solvent, and the number of the second surfactant molecules located in the vicinity of the surface of the solvent, to assess a suitability of a rinse solution composition to an extent by which collapse of the pattern can be prevented.

In addition, by using the ratio of the number of the first surfactant molecules located in the vicinity of the interface between the photoresist pattern 210 and the solvent 220b, and the number of the second surfactant molecules located on the surface of the solvent 220b, a competitive adsorption the additive molecules 240b, and the surfactant molecules 230b to the photoresist pattern 210 can be determined. Accordingly, a compositional suitability of the rinse solution can be assessed more precisely.

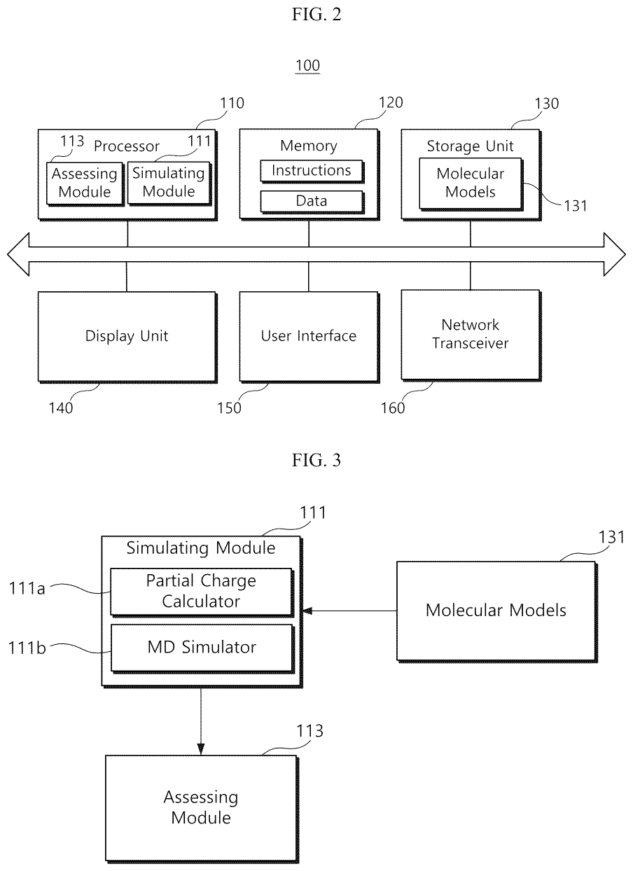

A configuration of the rinse solution assessing apparatus 100 will be described with reference to FIG. 2. FIG. 2 is a block diagram of a detailed configuration of the rinse solution assessing apparatus 100 according to an embodiment of FIG. 1.

In the rinse solution assessing apparatus 100 according to an embodiment of the present disclosure, a simulating module 111 of the processor 110 performs a molecular dynamics (MD) simulation of a cell, in which are placed the photoresist molecules based on the photoresist molecular model, the surfactant molecules based on the surfactant molecular model, the additive molecules based on the additive molecular model, and the solvent molecules based on the solvent molecular model. An assessing module 113 of the processor 110 computes the number and/or ratio of the surfactant molecules at a plurality of locations of the cell, at which a molecular dynamics (MD) simulation is performed.

Referring to FIG. 2, in an embodiment, the rinse solution assessing apparatus 100 includes the processor 110, the memory 120, the storage unit 130, a display unit 140, a user interface 150, and a network transceiver 160.

Instructions that are loaded and temporarily stored in the memory 120 control an operation of the processor 110.

The storage unit 130 stores a plurality of different kinds of molecular models 131. The memory 120 stores simulation data that includes molecules based on one of the molecular models, a size of the cell in which the molecules are placed, the method by which the molecules are placed in the cell, an ensemble condition under which the molecular dynamics (MD) simulation is performed, etc. The photoresist molecules, the surfactant molecules, the additive molecules, and the solvent molecules loaded into the memory 120 are data structures that can be computed. During a molecular dynamics (MD) simulation of the photoresist molecules, the surfactant molecules, the additive molecules, and the solvent molecules loaded into the memory 120, the processor 110 calculates locations and directions thereof in the cells.

The molecular model 131 includes chemoinformatics information. The molecular models 131 include molecular model information. For example, the molecular models 131 include information on the physical structure, such as the kinds of atoms that constitute the molecules and bonding angles of the atoms. According to an embodiment, the molecular models 131 include information on an energy distribution of electrons in an interior of the molecules. The electron energy distribution of the molecular model 131 can be calculated by a density functional theory (DFT) calculating module.

The storage unit 130 includes a computer-readable storage medium. The storage unit medium includes all kinds of recording devices that store computer-readable data. The storage unit medium may be at least one of a hard disk drive (HDD), a solid state disk (SSD), a silicon disk drive (SDD), an ROM, an RAM, a CD-ROM, a magnetic tape, a floppy disk, or an optical data storage unit device.

The simulating module 111 of the processor 110 performs a molecular dynamics (MD) simulation while changing at least some of the system conditions, such as the number of particles, a pressure, an energy, a volume, or a temperature, or by fixing them to specific values. At a given state of the system conditions, the simulating module 111 of the processor 110 calculates forces that are imparted to the molecules and calculates the behaviors of the molecules.

The simulating module 111 according to an embodiment of the present disclosure places the photoresist molecules in a cell having a preset spatial volume. The simulating module 111 performs a molecular dynamics (MD) simulation with the NPT ensemble in the cell, in which the photoresist molecules are placed, until a density of the photoresist pattern reaches a preset density condition. The density condition may a density decrease of the photoresist molecules within a preset range or that the density of the photoresist molecules converges to a constant value. The simulating module 111 performs a molecular dynamics (MD) simulation while maintaining the pressure and temperature of the cell, in which the photoresist molecules are placed, constant.

In an embodiment, the photoresist pattern reaches a preset density condition during a molecular dynamics (MD) simulation performed with the NPT ensemble in the cell in which the photoresist molecules are placed. For example, the density of the photoresist molecules located in the preset spatial area in the cell converges to a constant value due to the behaviors of photoresist molecules at a given pressure. In an embodiment, a molecular dynamics (MD) simulation is performed for a preset time period with the NPT ensemble in the cell in which the photoresist molecules are placed.

The simulating module 111 places the surfactant molecules, the additive molecules, and the solvent molecules in the cell at locations that are separated from the photoresist pattern, the density of which converges to a constant value.

The simulating module 111 places the surfactant molecules, the additive molecules, and the solvent molecules at random at locations in the cell that are separated from the photoresist by a preset minimum distance. For example, the simulating module 111 places the surfactant molecules, the additive molecules, and the solvent molecules at locations that are spaced apart from the photoresist pattern by at least 0.3 nm.

In an embodiment, the simulating module 111 sets a specific volume in the cell as a vacuum layer. For example, the simulating module 111 sets a vacuum layer with a thickness of at least 2 nm at an upper location opposite from the photoresist pattern.

The simulating module 111 performs a molecular dynamics (MD) simulation with the NVT ensemble in the cell in which are placed the photoresist patterns, the surfactant molecules, the additive molecules, and the solvent molecules. The simulating module 111 performs a molecular dynamics (MD) simulation while maintaining a constant temperature and a constant volume in the cell.

In an embodiment, the simulating module 111 perform a molecular dynamics (MD) simulation of the cell in which are placed the surfactant molecules, the additive molecules, and the solvent molecules, while fixing the molecules that constitute the photoresist pattern. The adsorption behavior of the surfactant molecules and the additive molecules on the photoresist patterns is simulated. As a result, the number of surfactant molecules and the number of additive molecules located in a vicinity of the interface between the photoresist pattern and the solvent and in a vicinity of the surface of the solvent can be computed.

According to an embodiment, the simulating module 111 performs a molecular dynamics (MD) simulation with the NVT ensemble for a preset time period in the cell in which are placed the photoresist patterns, the surfactant molecules, the additive molecules, and the solvent molecules. For example, the molecular dynamics (MD) simulation may be performed for at least 20 ns.

The assessing module 113 of the processor 110 according to an embodiment of the present disclosure performs a molecular dynamics (MD) simulation of the cell in which the molecules are placed, and then computes the number of each kind of molecule at a plurality of locations.

The assessing module 113 computes the number of the first surfactant molecules located in a vicinity of the interface between the photoresist pattern and the solvent, and the number of second surfactant molecules located in a vicinity of the surface of the solvent.

In an embodiment, after performing a molecular dynamics (MD) simulation for a predetermined time period, the assessing module 113 identifies and computes the number of the surfactant molecules located in a plurality of preset areas in a vertical direction from the photoresist pattern. The volume, in which the number of the surfactant molecules is calculated, is within a specific range from the interface between the photoresist pattern and the solvent, or within a specific range from the surface of the solvent. When a vacuum layer is set, the surface of the solvent is an interface between the solvent and the vacuum layer. The specific range, for example, may be 1 nm, but embodiments of the present disclosure are not necessarily limited thereto.

In an embodiment, the assessing module 113 computes a ratio of the number of surfactant molecules located in a volume within a specific range from the interface between the photoresist pattern and the solvent, and the number of surfactant molecules located in a volume within a specific range from the surface of the solvent. The ratio may be called a surfactant content ratio.

The rinse solution assessing apparatus 100 can change a kind or a concentration of any of the different photoresist molecular models, the different surfactant molecular models, the different additive molecular models, and/or the different solvent molecular models, and perform a molecular dynamics (MD) simulation under the same simulation conditions. For example, the rinse solution assessing apparatus 100 can individually simulate the photoresist material, the surfactant material, the additive material, and/or the solvent material, and can compute and compare the numbers and/or surfactant content ratios of the surfactant molecules at a plurality of locations of the cell.

Accordingly, in an embodiment, the adsorption behavior of the surfactant on the photoresist pattern can be analyzed for different materials, and a surfactant material can be selected that can lower a possibility of collapse due to dissolution of the photoresist pattern. In an embodiment, based on the simulation result, a ratio of the surfactant and the additive in the rinse liquid, or the additive material or the solvent material, can be selected.

The simulating module 111 and/or the assessing module 113 can be implemented in one of software, hardware, and/or firmware, or a combination thereof. When a part or all of the simulating module 111 and/or the assessing module 113 are implemented as software, one or more instructions that constitute the simulating module 111 and/or the assessing module 113 are stored in the storage unit 130, or loaded into memory 120.

The display unit 140 displays an assessment result based on the simulation result of the rinse solution assessing apparatus 100. The display unit 140 is not limited to a visual display and can display a result based on sight, hearing, or tactile sensation. For example, the display unit 140 can compare and display a surfactant content ratio that is computed based on the number of the surfactant molecules at a plurality of locations of the cell of different photoresist materials, different surfactant materials, different additive materials, and/or different solvent materials and/or based on a simulation results.

The user interface 150 can receive an input from a user to control the rinse solution assessing apparatus 100. The user interface 150 can receive an input from the user for an output that is displayed on the display unit 140. The user interface 150 may include a graphical user interface (GUI) displayed on the display unit 140 and a touch input means implemented on the display unit 140.

In an embodiment, the rinse solution assessing apparatus 100 receives the molecular models 131 online through the network transceiver 160. The network transceiver 160 includes at least one of a mobile communication module based on Long Term Evolution (LTE) or Long Term Evolution-Advanced (LTE-A), etc., a wireless Internet module based on Wi-Fi, WLAN, etc., Bluetooth™, radio frequency identification (RFID), infrared communication, ultra wideband (UWB), ZigBee, or near field communication (NFC).

FIG. 3 is a block diagram of the simulating module 111 of the rinse solution assessing apparatus 100, according to an embodiment.

In an embodiment, the simulating module 111 performs a molecular dynamics (MD) simulation of the cell, in which the molecules generated based on the molecular models 131 are placed, and provides the result to the assessing module 113.

In an embodiment, the simulating modules 111 include a partial charge calculator 111a and a molecular dynamics simulator 111b.

The partial charge calculator 111a calculates the partial charges of the computable molecules that are generated based on the molecular models 131. The partial charge calculator 111a calculates the partial charge of the molecules of each material based on density functional theory-based calculations.

In an embodiment, the partial charge calculator 111a calculates the partial charges of the atoms that constitute the photoresist molecule by a Becke, 3-parameter, Lee-Yang-Parr (b3lyp) density functional theory-based calculation. The LACV3P** basis set can be used for calculating the partial charges of the photoresist molecules. For example, geometric optimization of the photoresist molecules is performed by a b3lyp/LACV3P** density functional theory calculation.

Similarly, the partial charge calculator 111a calculates the partial charges of the surfactant molecules, the additive molecules, and/or the solvent molecules based on the density functional theory-based calculations.

The molecular dynamics simulator 111b calculates interactions between atoms of the molecules based on the partial charges of the molecules in the cell, and calculates behaviors of the molecules by applying a force field. For example, the force field is an optimized potential for liquid simulations (OPLS) force field. When the behaviors of molecules are calculated by using an OPLS force field, the total energy of the molecules is calculated based on bond distances of the atoms, bonding angles of the atoms, dihedral angles of the atoms, and interaction energy of the non-bonded atoms. The molecular dynamics simulator 111a performs molecular dynamics calculations by using various force fields, such as assisted model building with energy refinement (AMBER), consistent force field (CFF), chemistry at harvard molecular mechanics (CHARMM), consistent-valence forcefield (CVFF), and Interface force filed (IFF), in addition to an OPLS force field, depending on the photoresist material, the surfactant material, and/or the additive material.

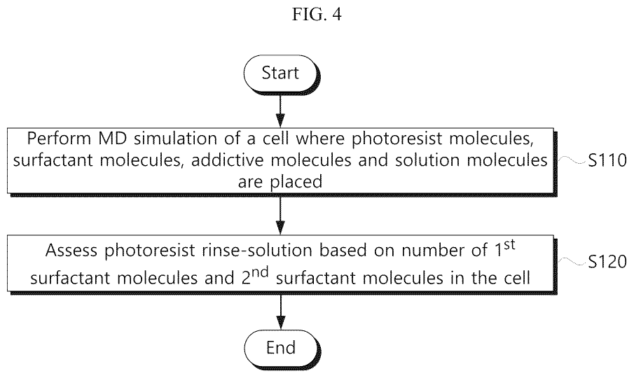

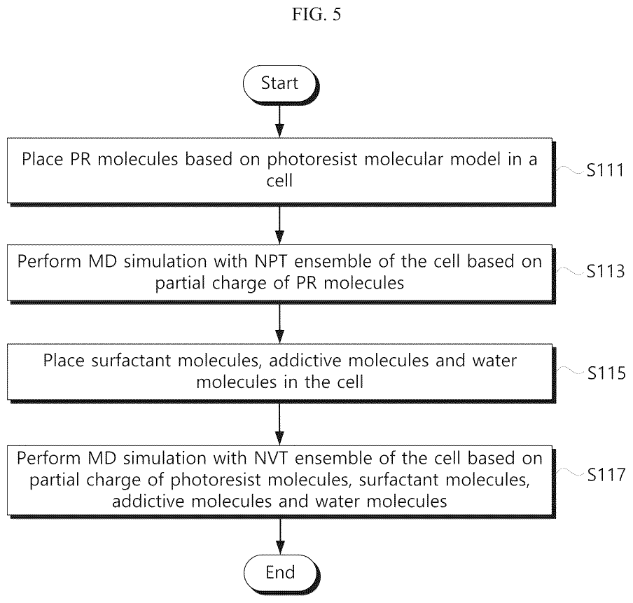

FIGS. 4 and 5 are flowcharts of a method for assessing a rinse solution according to an embodiment of the present disclosure. A method for assessing a rinse solution according to an embodiment of FIGS. 4 and 5 can be performed by the rinse solution assessing apparatus 100 according to the embodiment of FIG. 1 or a similar computing device.

Referring to FIG. 4, in an embodiment, in operation S110, the rinse solution assessing apparatus performs a molecular dynamics (MD) simulation of the cell in which are placed the photoresist molecules, the surfactant molecules, the additive molecules, and the solvent molecules. The solvent molecules may be water molecules.

According to an embodiment, FIG. 5 illustrates operation S110 in more detail.

Referring to FIG. 5, in an embodiment, the rinse solution assessing apparatus 100 according to an embodiment of the present disclosure forms a photoresist pattern in operations S111 and S113.

In operation S111, the rinse solution assessing apparatus places the photoresist molecules based on the photoresist molecular model in a first cell having a preset spatial volume in the simulation space. Horizontal and vertical sizes of the first cell are set in advance, and a height thereof is set to be automatically adjusted according to a change in a density value thereof according to the size and the pressure of the photoresist molecules. For example, the height of the first cell is automatically adjusted to be suitable for a density of 0.5 g/cm, depending on the size of the photoresist molecules.

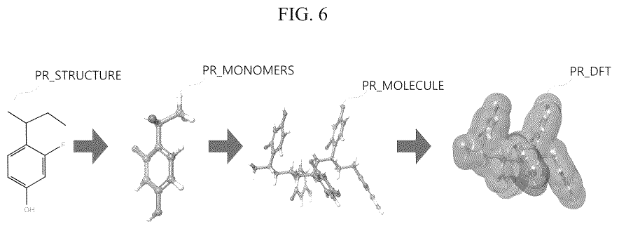

FIG. 6 illustrates a photoresist molecule on which a molecular dynamics simulation is performed based on the molecular model according to an embodiment of FIG. 5. Referring to FIG. 6, the rinse solution assessing apparatus generates a computable photoresist molecule PR_MOLECULE based on a photoresist monomer PR_MONOMER based on a photoresist structure PR_STRUCTURE that includes structural information, such as the atoms that constitute the photoresist molecule and the bonding angles of the atoms. For example, the rinse solution assessing apparatus generates the photoresist molecule PR_MOLECULE by combining a plurality of photoresist monomers PR_MONOMER.

The rinse solution assessing apparatus performs calculations based on density functional theory on the photoresist molecules PR_MOLECULE, and calculates the partial charges of the photoresist molecules PR_MOLECULE. The rinse solution assessing apparatus optimizes a structure of the photoresist molecule PR_MOLECULE based on the partial charges, and stores it as a photoresist DFT structure PR_DFT. In some embodiments, the photoresist molecule PR_MOLECULE with an optimized structure is stored as separate information from the partial charges.

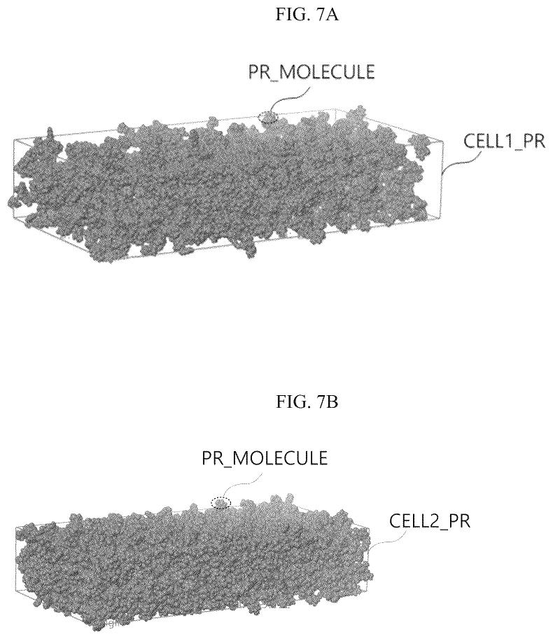

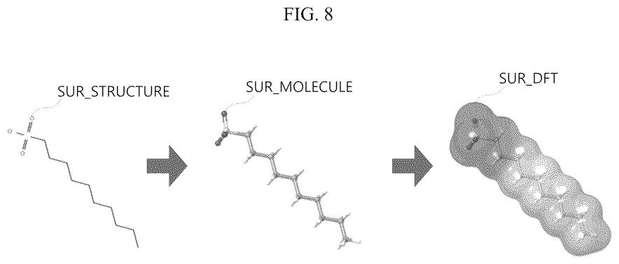

FIGS. 7A and 7B illustrate formation of a photoresist pattern in a molecular dynamics (MD) simulation according to an embodiment of FIG. 5.

In operation S113, referring to FIGS. 5 and 7A, in an embodiment, the rinse solution assessing apparatus performs a molecular dynamics (MD) simulation with the NPT ensemble in a first cell CELL1_PR in which the photoresist molecules PR_MOLECULE are placed. The rinse solution assessing apparatus simulates the molecular-level behaviors of photoresist molecules PR_MOLECULE by applying a force field. For example, the force field is an OPLS force field, but embodiments of the present disclosure are not necessarily limited thereto. The rinse solution assessing apparatus performs molecular dynamics (MD) simulations of the photoresist molecules PR_MOLECULE for a preset time period. For example, the molecular dynamics (MD) simulation is performed for 5 ns, but embodiment of the present disclosure are not necessarily limited thereto. In some embodiments, the rinse solution assessing apparatus performs the molecular dynamics (MD) simulations until a density value of photoresist molecules PR_MOLECULE satisfies a preset condition. For example, the rinse solution assessing apparatus performs the molecular dynamics (MD) simulation until a change in density value decreases to within a specific range, or until the density value converges to a specific value.

Referring to FIG. 7B, in an embodiment, a height of a first cell CELL2_PR, on which the molecular dynamics (MD) simulation is performed with the NPT ensemble, is decreased due to the behaviors of the photoresist molecules PR_MOLECULE. The photoresist molecules PR_MOLECULE in the first cell CELL2_PR, on which the molecular dynamics (MD) simulation is performed with the NPT ensemble, is called a photoresist pattern.

Referring to FIGS. 5 and 8, in an embodiment, in operation S115, the rinse solution assessing apparatus generates computable surfactant molecules SUR_MOLECULE based on a surfactant molecule structure SUR_STRUCTURE, similar to operation S111. In addition, the partial charges of surfactant molecules SUR_MOLECULE are calculated based on the density functional theory to generate surfactant DFT structure SUR_DFT. Similarly, additive molecules ADD_MOLECULE and solvent molecules SV_MOLECULE, which are computable, are generated. The partial charges of the additive molecules ADD_MOLECULE and the solvent molecules SV_MOLECULE are calculated.

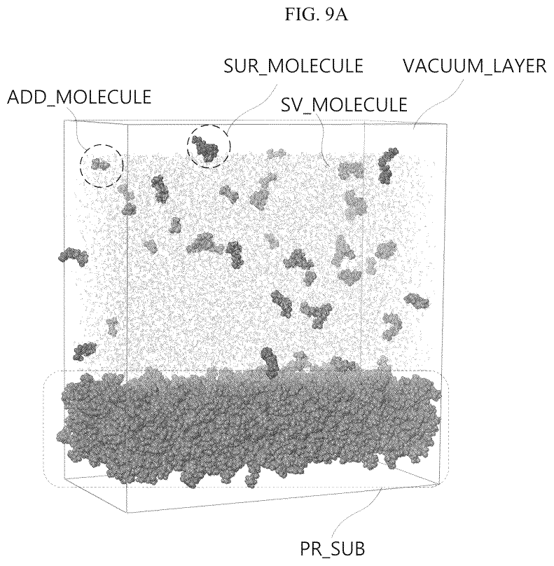

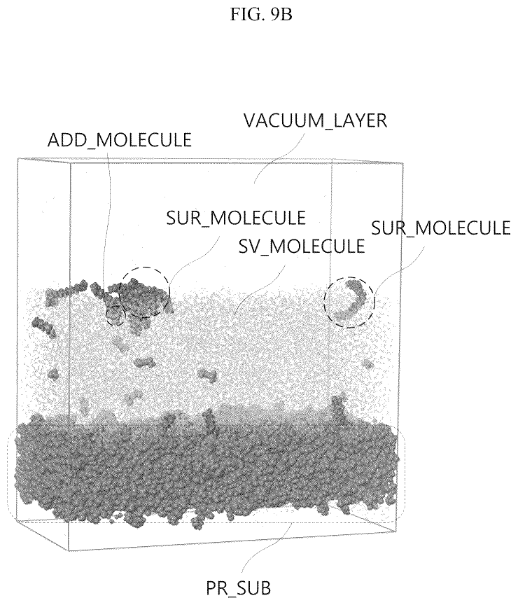

In operation S115, referring to FIG. 9A, in an embodiment, the rinse solution assessing apparatus spaces apart the surfactant molecules SUR_MOLECULE, the additive molecules ADD_MOLECULE, and the solvent molecules SV_MOLECULE from a photoresist pattern PR_SUB in the cell, in which the photoresist pattern PR_SUB is formed, and places them at random. In some embodiments, the cell in which the photoresist pattern PR_SUB is formed, is placed under another cell, and the surfactant molecules SUR_MOLECULE, the additive molecules ADD_MOLECULE, and the solvent molecules SV_MOLECULE are placed on the photoresist pattern PR_SUB. The solvent molecules SV_MOLECULE may be water molecules, or may include other materials according to an embodiment.

In an embodiment, the rinse solution assessing apparatus places a vacuum layer VACUUM_LAYER on an upper layer opposite from the photoresist pattern PR_SUB of the surfactant molecules SUR_MOLECULE, the additive molecules ADD_MOLECULE, and the solvent molecules SV_MOLECULE.

Referring to FIG. 5, in an embodiment, in operation S117, the rinse solution assessing apparatus performs a molecular dynamics (MD) simulation with the NVT ensemble of the first cell, in which the photoresist pattern PR_SUB is formed. Referring to FIGS. 9A and 9B, the molecular-level behavior is simulated based on the partial charges of the surfactant molecules SUR_MOLECULE, the additive molecules ADD_MOLECULE, and the solvent molecules SV_MOLECULE.

In an embodiment, the rinse solution assessing apparatus performs a molecular dynamics (MD) simulation on the surfactant molecules SUR_MOLECULE, the additive molecules ADD_MOLECULE, and the solvent molecules SV_MOLECULE, based on different force fields. For example, the rinse solution assessing apparatus uses an OPLS-based force field for the surfactant molecules SUR_MOLECULE and the additive molecules ADD_MOLECULE, and another force field for the solvent molecules SV_MOLECULE. When the solvent molecules SV_MOLECULE are water molecules, a force field is applied that is based on a TIP4P model that accurately expresses a coordination structure of water. The following examples are illustratively described on a premise that the solvent molecules SV_MOLECULE are water molecules.

Referring to FIGS. 5 and 9A, in an embodiment, the rinse solution assessing apparatus performs a molecular dynamics (MD) simulation on the surfactant molecules SUR_MOLECULE, the additive molecules ADD_MOLECULE, and the solvent molecules SV_MOLECULE while the photoresist molecules in the photoresist pattern PR_SUB are fixed.

In operation S117, the rinse solution assessing apparatus performs a molecular dynamics (MD) simulation with the NVT ensemble for a preset time period. For example, the simulation is performed for 20 ns, but embodiments of the present disclosure are not necessarily limited thereto.

Referring to FIG. 9B, in an embodiment, after the molecular dynamics (MD) simulation is performed, the vacuum layer VACUMM_LAYER is expanded, some additive molecules ADD_MOLECULE are placed on the surfaces of the solvent molecules, and some other additive molecules ADD_MOLECULE are placed between the solvent molecules or in a vicinity of the photoresist pattern PR_SUB. The surfactant molecules SUR_MOLECULE are also similarly placed.

Referring again to FIG. 4, in an embodiment, in operation S120, the rinse solution assessing apparatus assesses the photoresist rinse solution based on the numbers of first and second surfactant molecules computed at a plurality of locations in the cell on which the molecular dynamics (MD) simulation is performed.

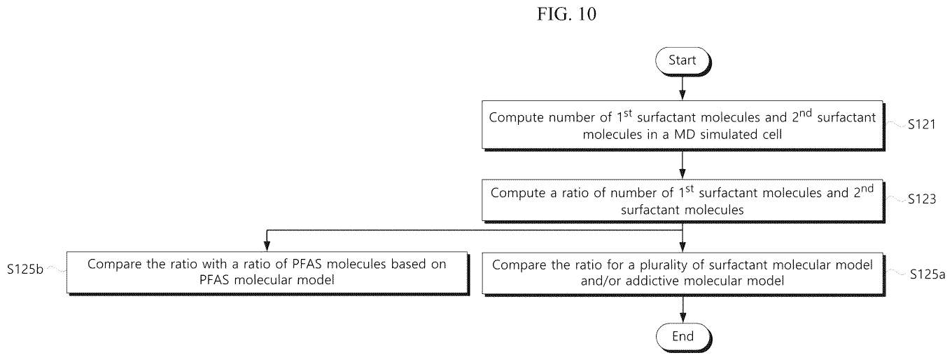

FIG. 10 is a flowchart of operation S120.

Referring to FIG. 10, in an embodiment, in operation S121, the rinse solution assessing apparatus computes the number of first surfactant molecules and second surfactant molecules at the plurality of locations in the cell on which the molecular dynamics (MD) simulation is performed.

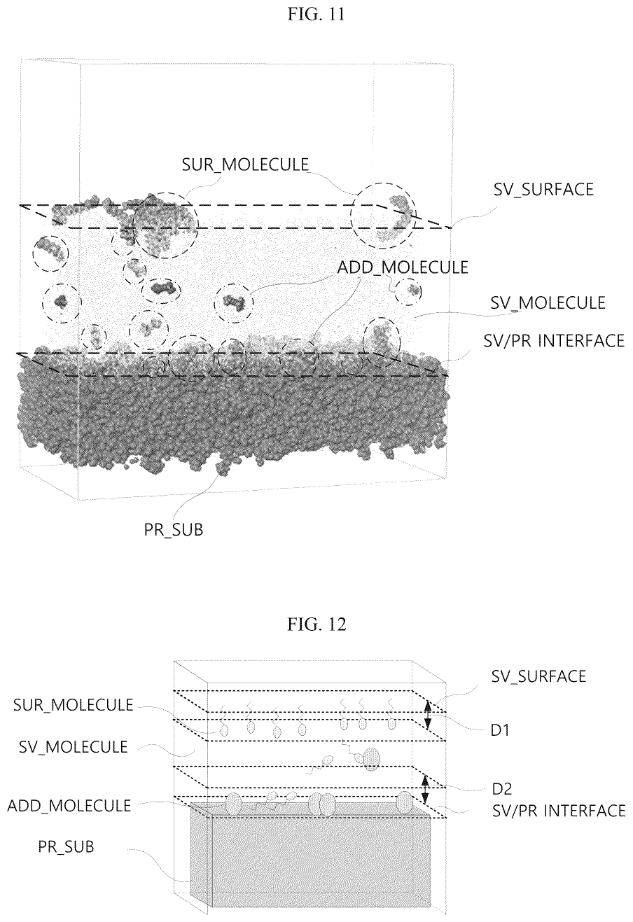

Referring to FIG. 11, in an embodiment, the first surfactant molecules SUR_MOLECULE are located in the vicinity of an interface SV/PR INTERFACE between the solvent molecules SV_MOLECULE and the photoresist pattern PR_SUB.

The second surfactant molecules SUR_MOLECULE are located in a vicinity of a surface SV_SURFACE of the solvent molecules SV_MOLECULE.

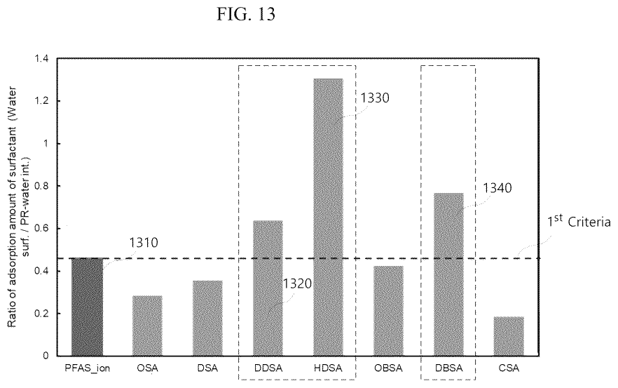

FIG. 12 further conceptualizes and illustrates a cell on which a molecular dynamics (MD) simulation is performed.

Referring to FIG. 12, in an embodiment, the rinse solution assessing apparatus computes the number of the surfactant molecules SUR_MOLECULE that are present within a preset distance D2 from the interface SV/PR INTERFACE as the number of the first surfactant molecules.

In addition, the rinse solution assessing apparatus computes the number of the surfactant molecules SUR_MOLECULE that are present within a preset distance D1 from the solvent surface SV_SURFACE as the number of the second surfactant molecules.

Referring to FIGS. 10 and 12, in an embodiment, in operation S123, the rinse solution assessing apparatus computes a surfactant content ratio as a ratio of the number of second surfactant molecules to the number of first surfactant molecules.

The rinse solution assessing apparatus assesses the photoresist rinse solution based on the calculated surfactant content ratio.

For example, in operation S125a, the rinse solution assessing apparatus compares the surfactant content ratio of a photoresist rinse solution that contains PFAS (perfluoroalkyl substances or Polyfluoroalkyl substances) and a photoresist rinse solution that contains the a surfactant other than PFAS. The rinse solution assessing apparatus assesses a photoresist rinse solution with a surfactant content ratio that is higher than that of the PFAS-containing photoresist rinse solution as having a lower possibility of collapse of the photoresist pattern. Accordingly, the rinse solution assessing apparatus determines the photoresist rinse solution that does not contain PFAS and has a low possibility of collapse of the photoresist pattern.

In another embodiment, the rinse solution assessing apparatus repeats the operations described above for different kinds of surfactant materials and/or different kinds of additive materials in operation S125b, and then compares the surfactant content ratios of the photoresist rinse solution based on the different materials. For example, the box for operation S125b in FIG. 10 compares PFAS molecules. The surfactant content ratio is used as a standard for the assessment of the photoresist rinse solution.

Similarly, the rinse solution assessing apparatus compares the surfactant content ratios by performing a molecular dynamics (MD) simulation on the photoresist rinse solutions with different ratios of the surfactant substances and the additive substances.

In some embodiments, the rinse solution assessing apparatus compares surfactant content ratios by performing a molecular dynamics (MD) simulation on photoresist rinse solutions with different concentrations of surfactant materials and additive materials in the solvent molecules.

FIG. 13 illustrates surfactant content ratios for different surfactant materials as an example. FIG. 13 illustrates the surfactant content ratios that are computed from simulation results based on PFAS, octane sulfonic acid (OSA), decane sulfonic acid (DSA), dodecane sulfonic acid (DDSA), hexadecane sulfonic acid (HDSA), octyl benzene sulfonic acid (OBSA), decyl benzene sulfonic acid (DBSA), and camphor-10-sulfonic acid (CSA) materials.

Referring to FIG. 13, in an embodiment, compared to a surfactant content ratio 1310 of the rinse solution that contains PFAS, a surfactant content ratio 1320 of the rinse solution that contains DDSA, a surfactant content ratio 1330 of the rinse solution that contains HDSA, and a surfactant content ratio 1340 of the rinse solution that contains DBSA are high. Accordingly, the rinse solution assessing apparatus determines DDSA, HDSA, and DBSA as surfactant candidate materials for the photoresist rinse solution.

In some embodiments, HDSA, which has the highest surfactant content ratio of rinse solutions that contain DDSA, HDSA, and DBSA, may be determined as the surfactant candidate material for the photoresist rinse solution.

In some embodiments, by using the surfactant content ratio as one of a plurality of assessment items, any one of DDSA, HDSA, or DBSA can be determined as the surfactant candidate material for the photoresist rinse solution.

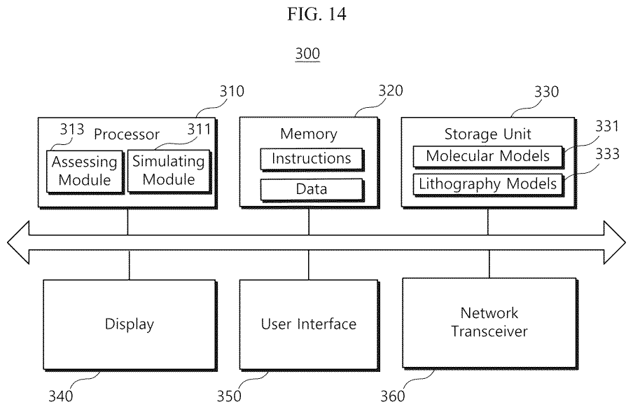

FIG. 14 is a block diagram of an apparatus that simulates a lithography process. A method for assessing photoresist rinse solutions according to an embodiment of the present disclosure is used as part of a simulation of the lithography process.

The lithography simulation apparatus is similar to the rinse solution assessing apparatus 100 described with reference to FIG. 2. Thus, a repeated detailed description of parts that are similar to those described above may be omitted.

A simulating module 311 of a processor 310 of a lithography simulation apparatus 300 according to an embodiment of the present disclosure simulates a photoresist pattern based on a lithography model. The simulating module 311 simulates the rinse process based on a simulated photoresist pattern. A simulation of a rinse process is a molecular dynamics (MD) simulation of the cell, in which are placed photoresist molecules based on a photoresist molecular model, surfactant molecules based on a surfactant molecular model, additive molecules based on an additive molecular model, and solvent molecules based on a solvent molecular model. An assessing module 313 of the processor 310 computes the numbers and/or ratios of surfactant molecules at a plurality of locations in the cell on which a molecular dynamics (MD) simulation is performed.

Referring to FIG. 14, in an embodiment, the lithography simulation apparatus 300 includes the processor 310, a memory 320, a storage unit 330, a display 340, a user interface 350, and a network transceiver 360.

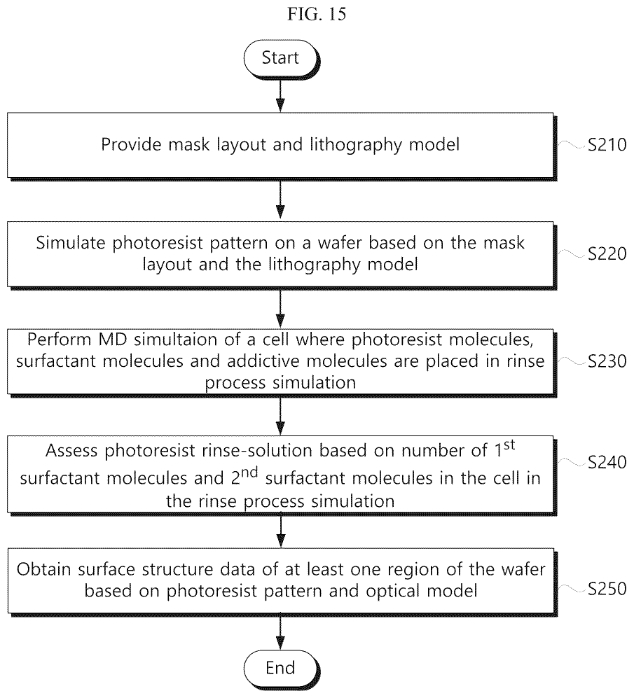

The lithography simulation apparatus 300 performs a simulation of the lithography process based on the lithography process simulation method of FIG. 15.

Referring to FIGS. 14 and 15, in an embodiment, a method for simulating a lithography process of the lithography simulation apparatus 300 will be described.

The storage unit 330 of the lithography simulation apparatus 300 according to an embodiment of the present disclosure includes molecular models 331 and lithography models 333.

The molecular models 331 include information on the molecular structures of at least one type of surfactant molecule and at least one type of additive molecule.

The lithography models 333 include at least one of an optical model, a resist model, a process window model, a defocus model, or a mask model.

The optical model describes formation of an aerial image by an exposure tool. The resist model describes an absorption of an incident aerial image by a photoresist and a development process that forms a final three-dimensional photoresist pattern. The process window model predicts a behavior and a size of a process window. The mask model describes effects of the kind and a size of the mask.

In operation S210, the lithography simulation apparatus 300 is provided with a mask layout and a lithography model. The mask layout and the lithography model may be provided through the network transceiver 360 or an offline storage device.

In operation S220, the simulating module 311 of the processor 310 is controlled by instructions of the memory 320 to load the mask layout and the lithography model 333 into the memory 320. The simulating module 311 simulates the photoresist pattern on a wafer based on the mask layout and the lithography model 333. The mask layout includes patterns that for printing integrated circuits on the wafer. The two dimensional shapes of cell patterns that are to be formed in a cell array area of the wafer are defined by the patterns.

Optionally, the simulating module 311 simulates a deionized water (DeIonized SV; DIW) washing process on the simulated photoresist pattern.

In operation S230, the simulating module 311 simulates a rinse process on the simulated photoresist pattern. Optionally, the simulating module 311 simulates the rinse process on the photoresist pattern, on which the deionized water washing process has been simulated. A simulation of the rinse process includes performing a molecular dynamics (MD) simulation on the molecular models 331.

The simulating module 311 performs a molecular dynamics (MD) simulation on the surfactant molecules based on a molecular model of at least one surfactant material, on the additive molecules based on a molecular model of at least one additive material, and on the solvent molecules based on at least one solvent molecular model. The surfactant molecules, the additive molecules, and the solvent molecules are computational data.

The simulating module 311 generates computational photoresist molecules of the photoresist material used in the simulated photoresist pattern. The simulating module 311 forms a photoresist pattern by performing a molecular dynamics (MD) simulation on the photoresist molecules. The simulating module 311 forms the photoresist pattern by performing a molecular dynamics (MD) simulation with the NPT ensemble on the photoresist molecules.

The simulating module 311 places the surfactant molecules, the additive molecules, and the solvent molecules in the cell in which the photoresist pattern is placed such that they are spaced apart from the photoresist pattern. The simulating module 311 performs a molecular dynamics (MD) simulation on cells in which the surfactant molecules, the additive molecules, and the solvent molecules are placed. The molecular dynamics (MD) simulation that is performed on the surfactant molecules, the additive molecules, and the solvent molecules is performed with the NVT ensemble. While the simulation is performed with the NVT ensemble, the photoresist molecules in the photoresist pattern are fixed.

In operation S240, the assessing module 313 computes the number of surfactant molecules at a plurality of locations in the cell after the molecular dynamics (MD) simulation of the surfactant molecules, the additive molecules, and the solvent molecules has ended. The plurality of locations at which the number of surfactant molecules is computed is similar to those described with reference to FIGS. 11 and 12.

In an embodiment, the assessing module 313 computes the ratio of the numbers of the surfactant molecules that are computed at the plurality of locations.

In operation S250, the simulating module 311 modifies the photoresist pattern simulated in operation S220 based on the ratio of the numbers of the surfactant molecules that are calculated at the plurality of positions. For example, the simulating module 311 determines a collapse degree of the photoresist pattern based on the ratio of the numbers of the surfactant molecules. The simulating module 311 modifies a shape of the photoresist pattern based on the collapse degree of the photoresist pattern.

The assessing module 313 acquires a surface structure from at least one area of the wafer based on the photoresist pattern modified by the rinse solution and the optical model.

The lithography simulation apparatus 300 determines whether to select a photoresist cleaning solution used in simulation of the rinsing process based on the acquired surface structure. For example, the surfactant used in the simulation of the rinse process is selected, a composition ratio of the surfactants and additives is selected, or the solvent is selected.

Accordingly, by combining a lithography process simulation with a molecular dynamics (MD) simulation of the photoresist cleaning solution, the photoresist pattern and/or the wafer surface structure due to the lithography process can be precisely simulated.

According to embodiments of the present disclosure, the rinse solution and/or the composition of the rinse solution that can prevent a collapse of the photoresist in the rinse process of the lithography, can be determined.

The above-described contents are detailed examples for carrying out embodiments of the present disclosure. In addition to the above-described embodiments, the present disclosure also includes embodiments that are simply designed or may be easily changed. In addition, embodiments of the present disclosure also include technologies that may be easily modified and implemented by using the embodiments. Therefore, the scope of embodiments of the present disclosure should not be limited to the above-described embodiments, but should be determined by the patent claims and the equivalents thereof, as well as the patent claims that will be presented below.

What is claimed is:

1. An apparatus for assessing a photoresist rinse solution, the apparatus comprising:
a processor;
a memory electrically connected to the processor and that stores instructions performed by the processor; and
a storage unit that stores a photoresist molecular model, a surfactant molecular model, an additive molecular model, and a solvent molecular model,
wherein the processor is configured by the instructions to:
perform a molecular dynamics simulation of a cell in which are placed photoresist molecules based on the photoresist molecular model, surfactant molecules based on the surfactant molecular model, additive molecules based on the additive molecular model, and solvent molecules based on the solvent molecular model; and
compute a number of first surfactant molecules located in the cell in a vicinity of an interface between a photoresist pattern that includes the photoresist molecules and a solvent that includes the solvent molecules, and a number of second surfactant molecules located in the cell in a vicinity of a surface of the solvent.

2. The apparatus of claim 1, wherein the processor is configured by the instructions to:
calculate partial charges of the photoresist molecules, the surfactant molecules, the additive molecules, and the solvent molecules, and perform a molecular dynamics simulation of the cell based on the partial charges.

3. The apparatus of claim 1, wherein the processor is configured by the instructions to:
place the photoresist molecules based on the photoresist molecular model in an interior of the cell, and form the photoresist pattern based on the photoresist molecules; and
place the surfactant molecules and the additive molecules based on the surfactant molecular model and the additive molecular model, respectively, together with the solvent molecules in the interior of the cell.

4. The apparatus of claim 3, wherein the processor is configured by the instructions to:
perform a molecular dynamics simulation of the cell with an isothermal-isobaric (NPT) ensemble with reference to a preset density in relation to a density of the photoresist pattern.

5. The apparatus of claim 1, wherein the processor is configured by the instructions to:
perform a molecular dynamics simulation of the cell with a canonical (NVT) ensemble based on the photoresist pattern, until a density of which reaches a preset density condition.

6. The apparatus of claim 1, wherein the processor is configured by the instructions to:
compute a ratio of the number of second surfactant molecules to the number of first surfactant molecules.

7. The apparatus of claim 6, wherein the processor is configured by the instructions to:
compare surfactant content ratios based on different kinds of surfactant molecular models or different kinds of additive molecular models.

8. The apparatus of claim 6, wherein the processor is configured by the instructions to:
compare the surfactant content ratio with a preset reference, wherein the reference is based on a number of first PFAS (perfluoroalkyl substances or polyfluoroalkyl substances) molecules located between the interface between the solvent and the photoresist pattern, and a number of second PFAS molecules located on the surface of the solvent.

9. A method performed by a computing device, the method comprising:
performing a molecular dynamics simulation of a cell in which are placed photoresist molecules, surfactant molecules, additive molecules, and solvent molecules; and
assessing a number of first surfactant molecules located on an interface between a photoresist pattern based on the photoresist molecules and a solvent based on the solvent molecules, and a number of second surfactant molecules located on a surface of the solvent.

10. The method of claim 9, comprising:
calculating partial charges of the photoresist molecules, the surfactant molecules, the additive molecules, and the solvent molecules, and performing a molecular dynamics simulation of the cell based on the partial charges.

11. The method of claim 9, comprising:
placing the photoresist molecules based on a photoresist molecular model in an interior of the cell, and generating the photoresist pattern based on the photoresist molecules; and
placing the surfactant molecules and the additive molecules based on a surfactant molecular model and an additive molecular model, respectively, together with the solvent molecules in the interior of the cell.

12. The method of claim 11, comprising:
performing a molecular dynamics simulation of the cell with an isothermal-isobaric (NPT) ensemble with reference to a preset density in relation to a density of the photoresist pattern.

13. The method of claim 12, comprising:
separating the surfactant molecules and the additive molecules from the photoresist pattern, together with the solvent molecules when the density of the photoresist pattern reaches a preset density condition, and
placing the separated surfactant molecules, the separated additive molecules, and the separated solvent molecules in the interior of the cell.

14. The method of claim 9, wherein performing the molecular dynamics simulation of the cell includes:
performing a molecular dynamics simulation of the cell with a canonical (NVT) ensemble based on the photoresist pattern until a density of which reaches a preset density condition.

15. The method of claim 9, comprising:
determining a ratio of the number of second surfactant molecules to the number of first surfactant molecules.

16. The method of claim 15, comprising:
comparing a surfactant content ratio based on different kinds of surfactant molecules or different kinds of additive molecules.

17. The method of claim 15, comprising:
comparing surfactant content ratios with a preset reference.

18. The method of claim 17, wherein the reference is based on a number of first PFAS (perfluoroalkyl substances or polyfluoroalkyl substances) molecules located between an interface between the solvent and the photoresist pattern, and a number of second PFAS molecules located on a surface of the solvent.

19. A method for simulating a lithography process that is performed by a computing device, the method comprising:
receiving a mask layout and a lithography model;
simulating a photoresist pattern on a wafer based on the mask layout and the lithography model;
simulating a deionized water washing process on the photoresist pattern;
simulating a rinse process on the photoresist pattern; and
acquiring surface structure data on one area of the wafer based on the photoresist pattern on which the rinse process is simulated and the lithography model, wherein simulating the rinse process includes:
forming a cell that includes photoresist molecules based on the photoresist pattern, placing surfactant molecules, additive molecules, and solvent molecules in the cell, and performing a molecule dynamics simulation of the cell; and
simulating a change in the photoresist pattern based on a number of first surfactant molecules located on an interface between a photoresist pattern based on the photoresist molecules and a solvent based on the solvent molecules, and a number of second surfactant molecules located on a surface of the solvent.

20. The method of claim 19, comprising:
simulating a change in the photoresist pattern based on different kinds of surfactant molecules or different kinds of additive molecules.
